(19) United States
(12) Patent Application Publication
(10) Pub. No.: US 2025/0205369 A1
(43) Pub. Date: Jun. 26, 2025

(54) DENDRIMER COMPOSITIONS AND USE IN TREATMENT OF NEUROLOGICAL AND CNS DISORDERS

(71) Applicants: THE JOHNS HOPKINS UNIVERSITY, Baltimore, MD (US); KENNEDY KRIEGER INSTITUTE, INC., Baltimore, MD (US)

(72) Inventors: Kannan RANGARAMANUJAM, Highland, MD (US); Sujatha KANNAN, Highland, MD (US); Elizabeth NANCE, Baltimore, MD (US); Mary E. BLUE, Baltimore, MD (US); Michael V. JOHNSTON, Baltimore, MD (US); William BAUMGARTNER, Ruxton, MD (US); Fan ZHANG, Baltimore, MD (US); Mary Ann WILSON, Columbia, MD (US); Barbara SLUSHER, Kingsville, MD (US)

(21) Appl. No.: 18/774,198

(22) Filed: Jul. 16, 2024

Related U.S. Application Data
(63) Continuation of application No. 15/502,744, filed on Feb. 8, 2017, now abandoned.
(60) Provisional application No. 62/036,839, filed on Aug. 13, 2014.
(60) Provisional application No. 62/036,675, filed on Aug. 13, 2014.

(51) Int. Cl. A61K 49/00; A61K 9/00; A61K 31/19; A61K 31/198; A61K 47/59

(52) U.S. Cl. CPC A61K 49/0054; A61K 9/0019; A61K 31/19; A61K 31/198; A61K 47/595; A61K 49/0032

(57) ABSTRACT

A dendrimer formulation, such as a PAMAM dendrimer or a multiarm PEG polymeric formulation has been developed for systemic administration to the brain or central nervous system. In the preferred embodiment, the dendrimers are in the form of dendrimer nanoparticles comprising poly(amidoamine)(PAMAM) hydroxyl-terminated dendrimers covalently linked to at least one therapeutic, prophylactic or diagnostic agent for treatment of one or more symptoms of neurodegenerative, neurodevelopmental or neurological disorders such as Rett syndrome of autism spectrum disorders. D6 generation dendrimers provide significantly enhanced uptake into areas of brain injury, providing a means for diagnosis as well as drug delivery.

### CROSS REFERENCE TO RELATED APPLICATIONS

This application claims priority to U.S. Provisional Patent Applications No. 62/036,675, filed Aug. 13, 2014 and 62/036,839, filed Aug. 13, 2014, incorporated herein by reference.

### BACKGROUND OF THE INVENTION

Drug delivery to the brain and to the central nervous system (CNS) is difficult, especially when targeted delivery to specific cells in the CNS are desirable. The drugs and the delivery vehicles have to overcome the blood-brain barrier (BBB), move in the brain tissue, and localize in the target cells. Patients with neurological diseases, including Parkinson's disease, Alzheimer's disease, brain tumors, and most neurogenetic disorders, suffer from severe debilitating symptoms and lack of therapeutic options that provide curative treatment. Various strategies have been developed to manipulate or bypass the blood-brain barrier (BBB) [Jain, Nanomedicine (Lond), 2012. 7 (8): 1225-33; Wohlfart, et al., J Control Release, 2012. 161 (2): 264-273.], which is the primary barrier to systemic delivery to the brain. These approaches include local administration to the CNS [Patel, et al., Advanced Drug Delivery Reviews, 2012. 64 (7): 701-705) and reversible disruption of the BBB via focused ultrasound [Marquet et al PLOS One 2011; 6 (7): e22598; Downs et al PLOS One. 2015 May 6; 10 (5): e0125911] or chemical reagents [Kroll, et al., Neurosurgery, 1998. 42 (S): 1083-1099; discussion 1099-100.]. However, once beyond the BBB, the anisotropic and electrostatically charged extracellular matrix (ECM) found between brain cells has been widely recognized as another critical barrier [Thorne, et al. Proc Natl Acad Sci USA. 2006 April 4; 103 (14): 5367-72; Nance, et al., Sci Transl Med, 2012. 4 (149): 149ra119; Sykova, et al., Physiol Rev, 2008. 88 (4): 1277-1340; Zamecnik, J., Acta Neuropathol, 2005. 110 (5): 435-442]. This ‘brain tissue barrier’, regardless of administration method, hampers widespread distribution of macromolecules and nanoparticles in the brain, thereby limiting their coverage throughout the disseminated target area of neurological diseases [Voges, J., et al., Ann Neurol, 2003. 54 (4): 479-487; Nance, E. A., et al., Sci Transl Med, 2012. 4 (149): 149ra119; Sykova, et al., Physiol Rev. 2008. 88 (4): 1277-340; Mackay, et al., Brain Res, 2005. 1035 (2): 139-153]. The BCM is rich in hyaluronan, chondroitin sulfate, proteoglycans, link proteins and tenascins and may provide a negatively charged adhesive barrier to the penetration of cationic polymeric carriers [Sykova, et al., Physiol Rev. 2008. 88 (4): 1277-1340; Zimmermann, et al., Histochem Cell Biol, 2008. 130 (4): 635-653]. Moreover, the pore size of the ECM imposes a steric barrier for the movement of nanoparticles in the CNS with non-adhesive 114 nm, but not 200 nm, particles able to penetrate within the brain tissue [Nance, E. A., et al., Sci Transl Med, 2012. 4 (149): p. 149ra119; Kenny, G. D., et al., Biomaterials, 2013. 34 (36): 9190-9200. It has been shown that sub-100 nm nanoparticles exceptionally well-coated with hydrophilic and neutrally charged polyethylene glycol (PEG) rapidly diffuse in the brain ECM, allowing the widespread distribution of therapeutics [Nance et al., ACS Nano, 2014 Oct. 28;8 (10): 10655-64. doi: 10.1021/nn504210g. Epub 2014 Oct. 8; Nance, E. A., et al., Sci Transl Med, 2012. 4 (149): p. 149ra119].

The accumulated knowledge of specific genetic targets that can alter or reverse the natural history of CNS diseases has rendered gene therapy an attractive therapeutic strategy [O'Mahony, A. M., et al., J Pharm Sci, 2013. 102 (10): 3469-3484; Lentz, et al., Neurobiol Dis, 2012. 48 (2): 179-188.]. Multiple preclinical and clinical studies have aimed to improve the delivery of nucleic acids to the CNS using leading viral or non-viral gene vectors with specific focus to enhancing the level and distribution of transgene expression throughout the brain tissue [O'Mahony, et al., J Pharm Sci, 2013. 102 (10): 3469-3484; Peroz-Martinez, et al., J Alzheimers Dis, 2012. 31 (4): 697-710].

Viral gene vectors, though relatively efficient, have been limited by one or more drawbacks, including low packaging capacity, technical difficulties in scale-up, high cost of production [Thomas, et al., Nat Rev Genet, 2003. 4 (5): 346-358.] and risk of mutagenesis [Olsen and Stein, N Engl J Med, 2004. 350 (21): 2167-2179.]. Furthermore, despite the immune privileged nature of the CNS, neutralizing immune responses may occur secondary to repeated administrations or prior exposures [Lentz, et al., Neurobiol Dis, 2012. 48 (2): 179-188; Xiao, X., et al., J Virol, 1996. 70 (11): 8098-8108; Chirmule, N., et al., J Virol, 2000. 74 (5): 2420-2425; Lowenstein, P. R., et al., Curr Gene Ther, 2007. 7 (S): p. 347-60; Lowenstein, P. R., et al., Neurotherapeutics, 2007. 4 (4): 715-724; Voges, J., et al., Ann Neurol, 2003. 54 (4): 479-487.].

Non-viral gene vectors can offer an attractive alternate strategy for gene delivery without many of these limitations [O'Mahony, A. M., et al., J Pharm Sci, 2013. 102 (10): 3469-3484]. Cationic polymer-based gene vectors provide a tailorable platform for DNA condensation and efficient gene transfer in vitro and in vivo. Their positive charge density allows for stable compaction of negatively charged nucleic acids [Sun, X. and N. Zhang, Mini Rev Med Chem, 2010, 10 (2): 108-125; Dunlap, D. D., et al., Nucleic Acids Res, 1997. 25 (15): 3095-3101] and protects them from enzymatic degradation [Kukowska-Latallo, J. F., et al., Hum Gene Ther, 2000. 11 (10): 1385-1395.], Also, the number of protonable amines provides increased buffering capacity that facilitates endosome escape via the “proton sponge effect”, leading to efficient transfection [Akinc, A., et al., J Gene Med, 2005. 7 (5): 657-663]. A wide variety of cationic polymers have been developed for this purpose, offering gene vectors with diverse physicochemical profiles and in vivo behaviors [Mintzer, M. A. and E. E. Simanek. Chem Rev, 2009, 109 (2): 259-302; Pathak, et al., Biotechnol J, 2009. 4 (11): 1559-72.]. However, non-viral gene vectors still face a number of barriers prior to reaching the target cells in the brain [O'Mahony, et al., J Pharm Sci, 2013. 102 (10): 3469-3484].

Convection enhanced delivery (CED) can be applied to further enhance the distribution of therapeutics by providing a pressure gradient during intracranial administration [Allard, et al., Biomaterials, 2009. 30 (12): 2302-2318.]. However, CED is unlikely to provide a significant benefit if particles remain entrapped in the brain parenchyma due to adhesive interactions and/or steric obstruction. Physicochemical properties of particles that allow unhindered diffusion in the brain parenchyma remain critical for achieving enhanced particle penetration following CED [Allard, et al., Biomaterials, 2009, 30 (12): 2302-18; Kenny, et al., Biomaterials, 2013. 34 (36): 9190-9200]. Even following CED, the interactions between positively charged particles and the negatively charged ECM confine cationic nanoparticles to the point of injection and perivascular spaces, and limit their penetration into the brain parenchyma [Mackay, et al., Brain Res, 2005. 1035 (2): 139-153; Kenny, et al., Biomaterials, 2013. 34 (36): 9190-9200; Writer, et al., J Control Release, 2012. 162 (2): p. 340-8.]. In addition to overcoming the blood brain barrier and diffusing in the brain parenchyma, a key challenge is targeting specific cells involved in the disease process, such as microglia and astrocytes that are involved in immune processes in the brain. This becomes especially critical in several neuroinflammatory, neurodevelopmental and neurodegenerative disorders where diffuse neuroinflammation is a key factor and where several regions in the CNS may be involved [Kannan S, et al., Sci Transl Med., 2012, 18:4 (130: 130fs8)].

In summary, drug and gene delivery to the brain is difficult because of the BBB, the brain microenvironment, and the diffuse nature of the neuroinflammation. As a result, many neurological disorders, especially neurodevelopmental, therefore have limited therapeutic options and limited technology development.

Rett syndrome (RTT) is one example of a debilitating neurodevelopmental disorder. RTT affects girls by slowing development followed by sudden regression in function, in children who initially appear normal. These children have loss of purposeful movements of hands, increased hand wringing, breathing difficulties, decreased brain growth, inability to walk/crawl, inability to speak, intellectual disability and seizures. Patients with RTT exhibit several features seen in autism and may be considered as a severe form of autism. Inflammation in the brain plays a key role in the pathogenesis and worsening of symptoms in children with RTT and autism. There is no cure available for these disorders.

In RTT, it is not known if the blood brain barrier or the brain microenvironment is the primary barrier to treatment, or if it is a combination of both, as is the case for most neurological diseases. Current therapies include anti-seizure medications and occupational therapy for motor disabilities. Targeted therapies that attenuate inflammation could have an impact in both Rett and in autism spectrum disorders. If systemically administered therapies to suppress cells involved in neuroinflammation could reach the brain, it could have significant implications in improving effectiveness, reducing side effects and costs.

It is therefore an object of the present invention to provide improved delivery, such as specific targeting of injured cells, and targeting multiple pathways in these cells in the brain and CNS at the same time.

It is a further object of the invention to provide means of treating neurological, neurodevelopmental, and neurodegenerative disorders of the brain and central nervous system, especially autism and RTT.

### SUMMARY OF THE INVENTION

A pharmaceutical composition including dendrimers delivering therapeutic, prophylactic and/or diagnostic agents can be administered systemically to reach target cells in the brain and central nervous system. In a preferred embodiment, the dendrimer composition is used to treat neurological, neurodevelopmental, and neurodegenerative disorders of the brain and CNS, including autism spectrum disorder and RTT. As demonstrated using a RTT mouse model; conjugation of an anti-inflammatory agent to the dendrimers results in significant improvement in mobility, gait, paw wringing, paw clenching, tremors and in respiratory patterns when compared to untreated or free drug treatment. The dendrimer conjugates are significantly better than the drug alone in improving mortality and motor/behavioral function, when compared to untreated animals. The dendrimers with the surface attributes described herein, overcome many current ‘brain tissue barrier’ related challenges.

In the preferred embodiment, the dendrimers are in the form of dendrimer nanoparticles comprising poly(amidoamine)(PAMAM) hydroxy)-terminated dendrimers covalently linked to at least one therapeutic, prophylactic or diagnostic agent. In a particularly preferred embodiment for treating RTT or autism spectrum disorders, dendrimer nanoparticles include one or more ethylene diamine-core PAMAM hydroxyl-terminated generation-4-10 (≥G4-OH) dendrimers covalently linked to a biologically active agent, in an amount effective to treat one or more symptoms of Rett syndrome or autism spectrum disorders in the subject. Excitotoxicity disorders may also be treated, using the same compositions.

Results demonstrate that significantly enhanced uptake by damaged or diseased brain is observed with generation-6 dendrimers as compared to generation-4 dendrimers. As described in the Examples, the generation-6 dendrimer is shown to have a highly desirable cerebrospinal fluid (CSF) to serum level in a large animal model of brain injury, indicating that these compositions are superior for delivering CNS drugs to the injured brain selectively. The positive results in a clinically-relevant large animal model (resembling humans in many aspects), underscores the importance of the findings. This provides a means for diagnosis as well as treatment. Another benefit of the dendrimers is that two or more different agents can be delivered using the same dendrimers. This may be two different therapeutic agents, or a combination of a therapeutic and one or more diagnostic or prophylactic agents.

### BRIEF DESCRIPTION OF THE DRAWINGS

FIG. 1A is a Kaplan-Meier survival curve following NAC and D-NAC therapy in MeCP2-null mice. Survival was assessed following twice weekly NAC(1) or D-NAC(2) therapy in MeCP2-null pops. D-NAC does not improve survival compared to non-treated animals (PBS, 3). D-NAC does improve safety of NAC. D-NAC and PBS treated MeCP2-null pups bad a significantly better 50% survival compared to NAC treated pups (p=0.014), indicating the potential toxicity of NAC when given as a free formulation. FIG. 1B is a line graph of neurobehavioral outcomes following D-NAC therapy in MeCP2-null mice, MeCP2-null mice were treated with saline (PBS, black dashed line (1)), 10 mg/kg NAC(red line (2)), or 10 mg/kg (on a NAC basis) D-NAC(blue line (3)) starting at 3 weeks of age (PND21). Pups were treated twice weekly. Behavior tests were performed at PND10 and PND17 to determine a baseline, and performed prior to treatment on each treatment day starting at PND21. Litter matched WT pups (solid black line (4)) were used as both weight and behavioral controls. D-NAC therapy significantly improved behavioral outcome compared to NAC and PBS treatments. D-NAC improved overall appearance of MeCP2-null mice compared to non-treated pups. Non-treated pups were emaciated, had multiple clenched paws, hunched posture, and poor eye condition.

FIGS. 2A-2F are graphs of the expression of Pro- and anti-inflammatory mRNA expression levels in WT (grey bars) and MeCP2-null (black bars) mice. FIG. 2A, TNF-α; FIG. 2B, IL-6; FIG. 2C, IL-Iβ; FIG. 2D, TGF-β; FIG. 2B, IL-10; and FIG. 2F, IL-4.

FIGS. 3A-C are graphs of the inflammatory profile in the brains of WT and pre-symptomatic and symptomatic MeCP2-null mice. mRNA levels of pro and anti-inflammatory cytokines were measured at ages 1, 2,3, 5, and 7 weeks old in the brains of WT (grey) and MeCP2-null (black) pups. Median 2ΔΔCT values are presented, and error bars are represented by the upper and lower interquartile range. (FIG. 3A) Changes in the inflammatory profile over time are presented as a ratio of a composite pro-inflammatory score, including TNFα, IL-6, and IL-1β, to a composite anti-inflammatory score, including TGF-β, IL-10, and IL-4. The composite score was generated by taking the median of all pro-inflammatory 2ΔΔCT values or all anti-inflammatory 2ΔΔCT values at each age for all pups at that age in a given genotype. (FIG. 3B) The pro-inflammatory profile in MeCP2-null mice trends towards an increase in pro-inflammatory markers at 2 weeks and 7 weeks. However, the anti-inflammatory mRNA expression (FIG. 3C) shows a significant decrease in MeCP2-null mice compared to age- and litter-matched WT mice at 2 weeks, 5 weeks, and 7 weeks of age. This suggests that the neuroinflammatory processes in the MeCP2-null mouse are driven by a significant decrease in anti-inflammatory expression, rather than a trend towards an increase in pro-inflammatory expression.

FIG. 4 is a graph of amount of D-Cy5 in brain (μg/g) as a function of severity of brain injury, based on composite behavioral score. This demonstration of correlation of uptake with severity of injury provides a means to diagnose the extent of injury.

FIG. 5 is a graph of the concentration of D-Cy5 in cerebral spinal fluid/concentration of D-Cy5 in serum over time in hours.

FIG. 6 is a graph of dendrimer accumulation (μg/g) in the hippocampus, cortex and cerebellum.

FIG. 7 is a graph of dendrimer accumulation (μg/g) in various organs and the brain.

### DETAILED DESCRIPTION OF THE INVENTION

### 1. Definitions

The term “therapeutic agent” refers to an agent that can be administered to prevent or treat one or more symptoms of a disease or disorder. Examples include, but are not limited to, a nucleic acid, a nucleic acid analog, a small molecule, a peptidomimetic, a protein, peptide, carbohydrate or sugar, lipid, or surfactant, or a combination thereof.

The term “treating” refers to preventing or alleviating one or more symptoms of a disease, disorder or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The phrase “therapeutically effective amount” refers to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

### II. Formulation

### A. Dendrimers

The term “dendrimer” as used herein includes, but is not limited to, a molecular architecture with an interior core, interior layers (or “generations”) of repeating units regularly attached to this initiator core, and an exterior surface of terminal groups attached to the outermost generation. Examples of dendrimers include, but are not limited to, PAMAM, polyester, polylysine, and PPI. The PAMAM dendrimers can have carboxylic, amine and hydroxyl terminations and can be any generation of dendrimers including, but not limited to, generation 1 PAMAM dendrimers, generation 2 PAMAM dendrimers, generation 3 PAMAM dendrimers, generation 4 PAMAM dendrimers, generation 5 PAMAM dendrimers, generation 6 PAMAM dendrimers, generation 7 PAMAM dendrimers, generation 8 PAMAM dendrimers, generation 9 PAMAM dendrimers, or generation 10 PAMAM dendrimers. Dendrimers suitable for use with include, but are not limited to, polyamidoamine (PAMAM), polypropylamine (POPAM), polyethylenimine, polylysine, polyester, iptycene, aliphatic poly(ether), and/or aromatic polyether dendrimers. Each dendrimer of the dendrimer complex may be of similar or different chemical nature than the other dendrimers (e.g., the first dendrimer may include a PAMAM dendrimer, while the second dendrimer may comprise a POPAM dendrimer). In some embodiments, the first or second dendrimer may further include an additional agent. The multiarm PEG polymer includes a polyethylene glycol having at least two branches bearing sulfhydryl or thiopyridine terminal groups; however, embodiments disclosed herein are not limited to this class and PEG polymers bearing other terminal groups such as succinimidyl or maleimide terminations can be used. The PEG polymers in the molecular weight 10 kDa to 80 kDa can be used.

A dendrimer complex includes multiple dendrimers. For example, the dendrimer complex can include a third dendrimer; wherein the third-dendrimer is complexed with at least one other dendrimer. Further, a third agent can be complexed with the third dendrimer. In another embodiment, the first and second dendrimers are each complexed to a third dendrimer, wherein the first and second dendrimers are PAMAM dendrimers and the third dendrimer is a POPAM dendrimer. Additional dendrimers can be incorporated without departing from the spirit of the invention. When multiple dendrimers are utilized, multiple agents can also be incorporated. This is not limited by the number of dendrimers complexed to one another.

As used herein, the term “PAMAM dendrimer” means poly(amidoamine) dendrimer, which may contain different cores, with amidoamine building blocks. The method for making them is known to those of skill in the art and generally, involves a two-step iterative reaction sequence that produces concentric shells (generations) of dendritic β-alanine units around a central initiator core. This PAMAM core-shell architecture grows linearly in diameter as a function of added shells (generations). Meanwhile, the surface groups amplify exponentially at each generation according to dendritic-branching mathematics. They are available in generations G0-10 with 5 different core types and 10 functional surface groups. The dendrimer-branched polymer may consist of polyamidoamine (PAMAM), polyglycerol, polyester, polyether, polylysine, or polyethylene glycol (PEG), polypeptide dendrimers.

In accordance with some embodiments, the PAMAM dendrimers used can be generation 4 dendrimers, or more, with hydroxyl groups attached to their functional surface groups. The multiarm PEG polymer comprises polyethylene glycol having 2 and more branches bearing sulfhydryl or thiopyridine terminal groups; however, embodiments are not limited to this class and PEG polymers bearing other terminal groups such as succinimidyl or maleimide terminations can be used. The PEG polymers in the molecular weight 10 kDa to 80 kDa can be used.

In some embodiments, the dendrimers are in nanoparticle form and are described in detail in international patent publication No. WO2009/046446.

### Preparation of PAMAM-NAC

Below is a synthetic scheme for conjugating N-acetylcysteine to an amine-terminated fourth generation PAMAM dendrimer (PAMAM-NH2), using N-succinimidyl 3-(2-pyridyldithio) propionate (SPDP) as a linker.

Synthesis of N-succinimidyl 3-(2-pyridyldithio) propionate (SPDP) is performed by a two-step procedure, Scheme 1. First. 3-mercaptopropionic acid is reacted by thiol-disulfide exchange with 2,2′-dipyridyl disulfide to give 2-carboxyethyl 2-pyridyl disulfide. To facilitate linking of amine-terminated dendrimers to SPDP, the succinimide group is reacted with 2-carboxyethyl 2-pyridyl disulfide to obtain N-succinimidyl 3-(2-pyridyldithio) propionate, by esterification with N-hydroxysuccinimide by using N,N′-dicyclohexylcarbodiimide and 4-dimethylaminopyridine.

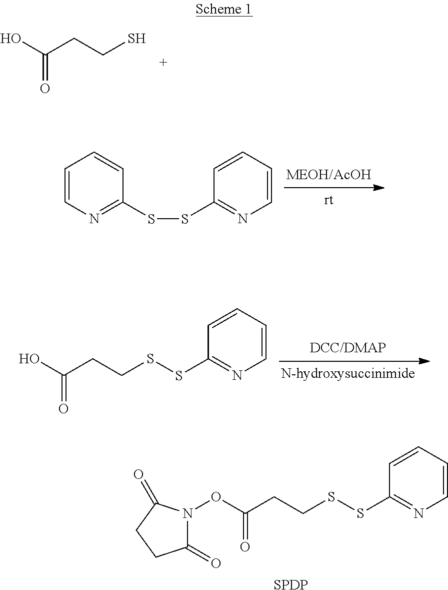

To introduce sulfhydryl-reactive groups, PAMAM-NH2 dendrimers are reacted with the heterobifunctional cross-linker SPDP, Scheme 2. The N-succinimidyl activated ester of SPDP couples to the terminal primary amines to yield amide-linked 2-pyridyldithiopropanoyl (PDF) groups, Scheme 2. After the reaction with SPDP, PAMAM-NH-PDP can be analyzed using RP-HPLC to determine the extent to which SPDP has reacted with the dendrimers.

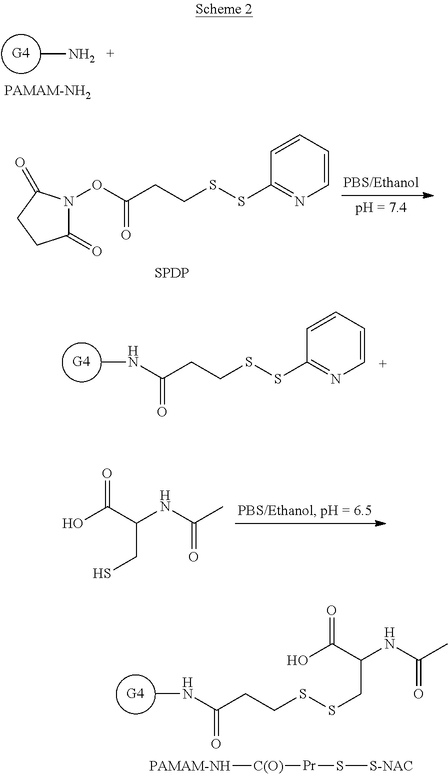

In another embodiment, the synthetic routes described in Scheme 4, below, can be used in order to synthesize D-NAC up to the pyridyldithio (PDP)-functionalized den crimes 3. Compound 3 is then reacted with NAC in DMSO, overnight at mom temperature to obtain. D-NAC 5.

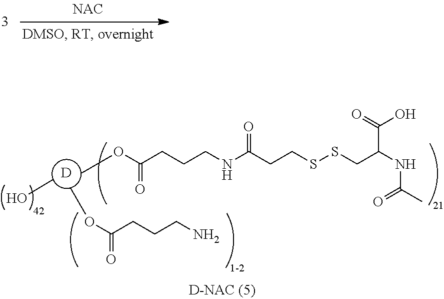

Initially, valproic acid is functionalized with a thiol-reactive group. A short PEG-SH having three repeating units of (CH₂)₂O—is reacted with valproic acid using DCC as coupling reagent as shown in Scheme 3. The crude PEG-VPA obtained is purified by column chromatography and characterized by proton NMR. In the NMR spectrum, there was a down-shift of the peak of CH; protons neighboring to OH group of PBG to 4.25 ppm from 3.65 ppm that confirmed the formation of PEG-VPA. Although the thiol group also may be susceptible to reacting with acid functionality, the NMR spectra did not indicate any downward shift of the peak belonging to CH₂ protons adjacent to thiol group of PEG. This suggests that the thiol group is free to react with the thiol-reactive functionalized dendrimer.

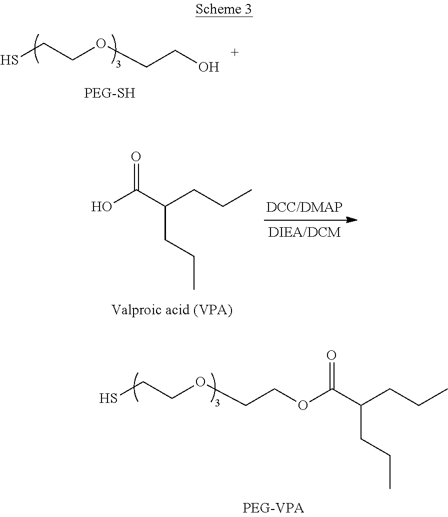

To conjugate PEG-VPA to the PAMAM-OH, a disulfide bond is introduced between the dendrimer and valproic acid, Scheme 4. First the dendrimer is converted to a bifunctional dendrimer 1 by reacting the dendrimer with fluorenylmethyloxycarbonyl (Fmoc) protected γ-aminobutyric acid (GABA). Conjugation of PEG-VPA to the bifunctional dendrimer involved a two-step process: the first step is the reaction of amine-functionalized bifunctional dendrimer 1 with N-succinimidyl-3-(2-pyridyldithio)-propionate (SPDP), and the second step involves conjugating the thiol-functionalized valproic acid. SPDP is reacted with the intermediate 2 in the presence of N,N-diisopropylethylamine (DIEA) to obtain pyridyldithio (PDP)-functionalized dendrimer 3.

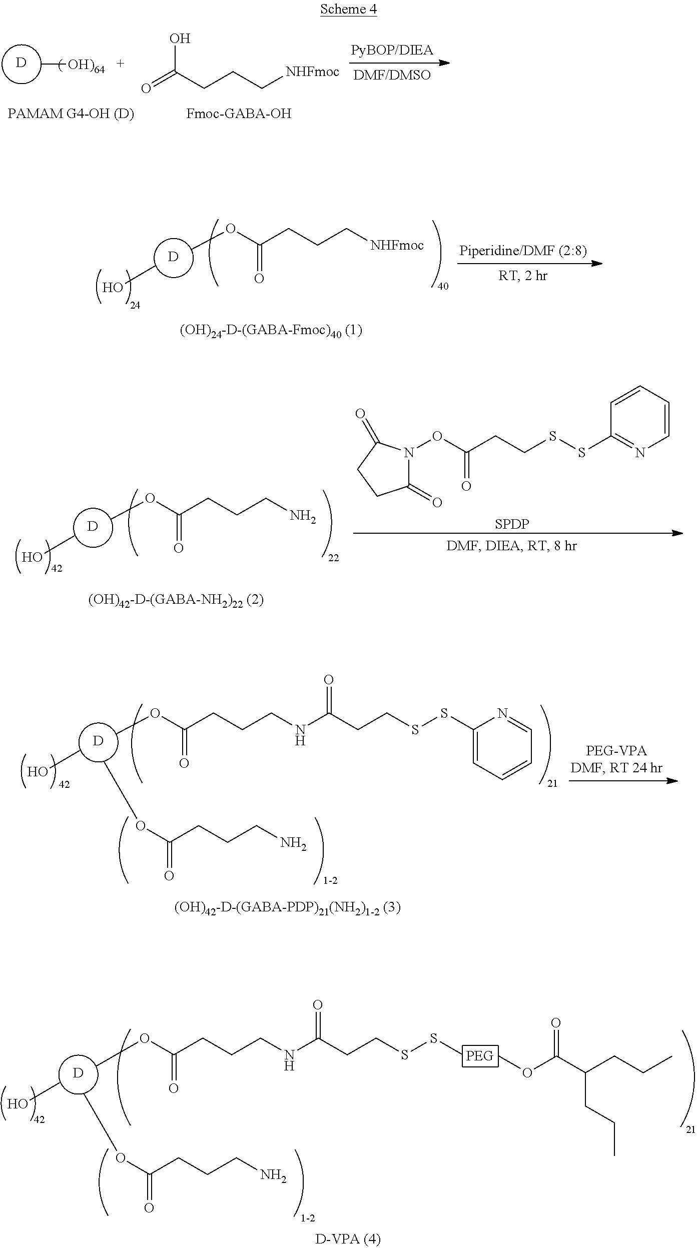

Even though this is an in situ reaction process, the structure was established by ¹H NMR. In the spectrum, new peaks between 6.7 and 7.6 ppm for aromatic protons of pyridyl groups confirmed the formation of the product. The number of pyridyl groups and number of GABA linkers were verified to be the same, which indicates that most of the amine groups reacted with the SPDP. Since this is a key step for the conjugation of the drug to the dendrimer, the use of mole equivalents of SPDP per amine group and time required for the reaction was validated. Finally, the PEG-VPA is reacted with the PDP-functionalized dendrimer in situ to get dendrimer-PEG-valproic acid (D-VPA). The formation of the final conjugate and loading of VPA were confirmed by ¹H NMR, and the purity of the conjugate was evaluated by reverse-phase HPLC. In the NMR spectrum, multiplets between 0.85 and 1.67 ppm for aliphatic protons of VPA, multiplets between 3.53 and 3.66 ppm for CH₂ protons of PEG, and absence of pyridyl aromatic protons confirmed the conjugate formation. The loading of the VPA is ˜21 molecules, estimated using a proton integration method, which suggests that 1-2 amine groups are left unreacted. In the HPLC chart, the elution time of D-VPA (17.2 min) is different from that for G4-OH (9.5 min), confirming that the conjugate is pure, with no measurable traces of VPA (23.4 min) and PEG-VPA (39.2 min). The percentage of VPA loading to the dendrimer is ˜12% w/w and validates the method for making gram quantities in three different batches.

### B. Coupling Agents and Spacers

Dendrimer complexes can be formed of therapeutically active agents or compounds (hereinafter “agent”) conjugated or attached to a dendrimer or multiarm PEG. The attachment can occur via an appropriate spacer that provides a disulfide bridge between the agent and the dendrimer. The dendrimer complexes are capable of rapid release of the agent in vivo by thiol exchange reactions, under the reduced conditions found in body.

The term “spacers” as used herein is intended to include compositions used for linking a therapeutically active agent to the dendrimer. The spacer can be either a single chemical entity or two or more chemical entities linked together to bridge the polymer and the therapeutic agent or imaging agent. The spacers can include any small chemical entity, peptide or polymers having sulfhydryl, thiopyridine, succinimidyl, maleimide, vinylsulfone, and carbonate terminations.

The spacer can be chosen from among a class of compounds terminating in sulfhydryl, thiopyridine, succinimidyl, maleimide, vinylsulfone and carbonate group. The spacer can comprise thiopyridine terminated compounds such as dithiodipyridine, N-Succinimidyl 3-(2-pyridyldithio)-propionate (SPDP), Succinimidyl 6-(3-[2-pyridyldithio]-propionamido) hexanoate LC-SPDP or Sulfo-LC-SPDP. The spacer can also include peptides wherein the peptides are linear or cyclic essentially having sulfhydryl groups such as glutathione, homocysteine, cysteine and its derivatives, arg-gly-asp-cys (RGDC), cyclo(Arg-Gly-Asp-d-Phe-Cys) (c(RGDfC), cyclo(Arg-Gly-Asp-D-Tyr-Cys), cyclo(Arg-Ala-Asp-d-Tyr-Cys). The spacer can be a mercapto acid derivative such as 3 mercapto propionic acid, mercapto acetic acid, 4 mercapto butyric acid, thiolan-2-one, 6 mercaptohexanoic acid, S mercapto valeric acid and other mercapto derivatives such as 2 mercaptoethanol and 2 mercaptoethylamine. The spacer can be thiosalicylic acid and its derivatives, (4-succinimidyloxycarbonyl-methyl-cx-2-pyridylthio) toluene, (3-[2-pyridithio]propionyl hydrazide, The spacer can have maleimide terminations wherein the spacer comprises polymer or small chemical entity such as bis-maleimido diethylene glycol and bis-maleimido triethylene glycol, Bis-Maleimidoethane, bismaleimidohexane. The spacer can comprise vinylsulfone such as 1,6-Hexane-bis-vinylsulfone. The spacer can comprise thioglycosides such as thioglucose. The spacer can be reduced proteins such as bovine serum albumin and human serum albumin, any thiol terminated compound capable of forming disulfide bonds The spacer can include polyethylene glycol having maleimide, succinimidyl and thiol terminations.

### C. Therapeutic, Prophylactic and Diagnostic Agents

The term “dendrimer complexes” as used herein refers to the combination of a dendrimer with a therapeutically, prophylactically and/or diagnostic active agent. The dendrimers may also include a targeting agent, but as demonstrated by the examples, these are not required for delivery to injured brain. These dendrimer complexes include an agent that is attached or conjugated to PAMAM dendrimers or multiarm PEG, which are capable of preferentially releasing the drug intracellularly under the reduced conditions found in vivo. The dendrimer complex, when administered by i.v. injection, can preferentially cross the blood brain barrier (BBB) only under diseased condition and not under normal conditions. The dendrimer complexes are also be useful for targeted delivery of the therapeutics in neuro-inflammation, cerebral palsy, ALS and other CNS diseases characterized by inflammation and damage to the tissues.

The agent can be either covalently attached or intra-molecularly dispersed or encapsulated. The dendrimer is preferably a PAMAM dendrimer up to generation 10, having carboxylic, hydroxyl, or amine terminations. The PEG polymer is a star shaped polymer having 2 or more arms and a molecular weight of 10 kDa to 80 kDa. The PEG polymer has sulfhydryl, thiopyridine, succinimidyl, or maleimide terminations. The dendrimer is linked to the agents via a spacer ending in disulfide, ester or amide bonds.

Representative therapeutic (including prodrugs), prophylactic or diagnostic agents can be peptides, proteins, carbohydrates, nucleotides or oligonucleotides, small molecules, or combinations thereof. Exemplary therapeutic agents include anti-inflammatory drugs, antiproliferatives, chemotherapeutics, vasodilators, and anti-infective agents. Antibiotics 2.5 include β-lactams such as penicillin and ampicillin, cephalosporins such as cefuroxime, cefaclor, cephalexin, cephydroxil, cefpodoxime and proxetil, tetracycline antibiotics such as doxycycline and minocycline, macrolide antibiotics such as azithromycin, erythromycin, rapamycin and clarithromycin, fluoroquinolones such as ciprofloxacin, enrofloxacin, ofloxacin, gatifloxacin, levofloxacin and norfloxacin, tobramycin, colistin, or aztreonam as well as antibiotics which are known to possess anti-inflammatory activity, such as erythromycin, azithromycin, or clarithromycin. A preferred anti-inflammatory is an antioxidant drug including N-acetylcysteine. Preferred NSAIDS include mefenamic acid, aspirin, Diflunisal, Salsalate, Ibuprofen, Naproxen, Fenoprofen, Ketoprofen, Deacketoprofen, Flurbiprofen, Oxaprozin, Loxoprofen, Indomethacin, Sulindac, Etodolac, Ketorolac, Diclofenac, Nabumetone, Piroxicam, Meloxicam, Tenoxicam, Droxicam, Lornoxicam, Isoxicam, Meclofenamic acid, Flufenamic acid, Tolfenamic acid, elecoxib, Rofecoxib, Valdecoxib, Parecoxib, Lumiracoxib, Etoricoxib, Firocoxib, Sulphonanilides, Nimesulide, Niflumic acid, and Licofelone.

Representative small molecules include steroids such as methyl prednisone, dexamethasone, non-steroidal anti-inflammatory agents, including COX-2 inhibitors, corticosteroid anti-inflammatory agents, gold compound anti-inflammatory agents, immunosuppressive, anti-inflammatory and anti-angiogenic agents, anti-excitotoxic agents such as valproic acid, D-aminophosphonovalerate, D-aminophosphonoheptanoate, inhibitors of glutamate formation/release, baclofen, NMDA receptor antagonists, salicylate anti-inflammatory agents, ranibizumab, anti-VEGF agents, including aflibercept, and rapamycin. Other anti-inflammatory drugs include nonsteroidal drug such as indomethacin, aspirin, acetaminophen, diclofenac sodium and ibuprofen. The corticosteroids can be fluocinolone acetonide and methylprednisolone. The peptide drug can be streptidokinase.

In some embodiments, the molecules can include antibodies, including, for example, daclizumab, bevacizumab (avastin®), ranibizumab (Lucentis®), basiliximab, ranibizumab, and pegaptanib sodium or peptides like SN50, and antagonists of NF.

Representative oligonucleotides include siRNAs, microRNAs, DNA, and RNA. The therapeutic agent can be a PAMAM dendrimer with amine or hydroxyl terminations,

Exemplary diagnostic agents include paramagnetic molecules, fluorescent compounds, magnetic molecules, and radionuclides, x-ray imaging agents, and contrast media. These may also be ligands or antibodies which are labelled with the foregoing or bind to labelled ligands or antibodies which are detectable by methods known to those skilled in the art.

Exemplary diagnostic agents include dyes, fluorescent dyes, Near infra-red dyes, SPECT imaging agents, PET imaging agents and radioisotopes. Representative dyes include carbocyanine, indocarbocyanine, oxacarbocyanine, thuicarbocyanine and merocyanine, polymethine, coumarine, rhodamine, xanthene, fluorescein, boron-dipyrromethene (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Floor 750, IRDye 800CW, IRDye 800RS, IRDye 700DX, ADS780WS, ADS830WS, and ADS832WS.

Representative SPECT or PET imaging agents include chelators such as di-ethylene tri-amine penta-acetic acid (DTPA), 1,4,7,10-tetra-azacyclododecane-1,4,7,10-tetraacetic acid (DOTA), di-amine dithiols, activated mercaptoacetyl-glycyl-glycyl-glycine (MAC3), and hydrazidonicotinamide (HYNIC).

Representative isotopes include Tc-94m, Tc-99m, In-111, Ga-67, Ga-68, Gd³⁺, Y-86, Y-90, Lu-177, Re-186, Re-188, Ca-64, Cu-67, Co-55, Co-57, F-18, Sc-47, Ac-225, Bi-213, Bi-212, Pb-212, Sm-153, Ho-166, and Dy-166.

Targeting moieties include folic acid, RGD peptides either linear or cyclic, TAT peptides, LERH and BH3.

In one embodiment for treating RTT and autism spectrum disorders the dendrimer nanoparticles are formed of PAMAM hydroxyl-terminated dendrimers covalently linked to at least one biologically active agent, in an amount effective to treat Rett syndrome and autism spectrum disorders in the subject.

The dendrimer complexes linked to a bioactive compound or therapeutically active agent can be used to perform several functions including targeting, localization at a diseased site, releasing the drug, and imaging purposes. The dendrimer complexes can be tagged with or without targeting moieties such that a disulfide bond between the dendrimer and the agent or imaging agent is formed via a spacer or linker molecule.

### D, Devices and Formulations

The dendrimers can be administered parenterally by subdural, intravenous, intra-amniotic, intraperitoneal, or subcutaneous routes.

The carriers or diluents used herein may be solid carriers or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The dendrimers can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

Formulations for convection enhanced delivery (“CED”) include solutions of low molecular weight sales and sugars such as mannitol.

### III. Methods of Treatment

### A. Delivery to the Brain and CNS

The dendrimer complex composition, including a dendrimer, preferably at least a fourth generation dendrimer and more preferably at least a six generation dendrimer, linked to a therapeutic, prophylactic or diagnostic agent, can selectively target microglia and astrocytes, which play a key role in the pathogenesis of several neurodegenerative diseases, including cerebral palsy. By targeting these cells, the dendrimers deliver agent specifically to treat neuroinflammation.

N-acetyl cysteine (“NAC”) has been extensively investigated and studied. It is also investigated for neuro-inflammation associated in maternal fetal infections. However, NAC suffers from low bioavailability due to high plasma protein binding. The dendrimer complex compositions overcome the plasma protein binding without affecting the activity of NAC.

G4 PAMAM-NAC can be ten to a hundred times more efficacious in vivo than the free drug NAC by single i.v. administration. The free drug NAC exhibits very high plasma protein binding resulting in reduced bioavailability. One of the major advantages of this dendrimer complex is that it enhances the bioavailability by restricting the unwanted drug plasma protein interactions and selectively results in rapid release of the drug intracellularly to exhibit the desired therapeutic action.

The high payload of the drug NAC in the G4 PAMAM-NAC requires very small quantities (˜50 mg/kg) of the carrier, PAMAM dendrimer, and smaller quantities of the drug (˜10 mg/kg), thereby reducing the amounts administered. In contrast, free NAC is typically used at 100-300 mg/kg daily doses in animal models. A decreased quantity of agent limits the side effects associated with the agent. Since the bioavailability of the agent remains high, the positive effects of the agent are not lowered despite the administration of smaller quantities of agent. The dendrimer complexes including the dendrimer-drug conjugates, restricts its biodistribution to tissues and organ and preferentially deliver the drug at the target site thereby reducing the undesired side effects.

Dendrimer complexes effectively transport across the BBB, and are therefore useful for targeted drug delivery in neurological, neurodevelopmental, and neurodegenerative disorders and brain injury, G4-PAMAM-S-S-NAC conjugates specifically target activated microglial cells and astrocytes in neuroinflammatory disorders:

The therapeutic efficacy of GA-PAMAM-S-S-NAC dendrimer conjugate was evaluated after two days of animal treatment with lipopolysaccharide (LPS) to induce white matter injury and hypomyelination in the developing rabbit brain (an animal model of Cerebral Palsy). NAC selectively delivered from the G4-PAMAM-S-S-NAC dendrimer complexes strongly suppressed pro-inflammatory cytokines (TNF-α, IL-6 mRNA); inflammatory signaling factors, including NE.kappa.B and nitrotyrosine, and enhanced OSH level. The G4-PAMAM-S-S-NAC was found to be ten to a hundred times more efficacious compared with free NAC. This supports a conclusion that the G4-PAMAM-S-S-NAC traversed across the BBB. The targeted delivery of NAC from dendrimer complex to active microglial cells improved the motor deficits and attenuated recovery from the LPS-induced brain injury in a neonatal rabbit model of cerebral palsy.

A significant reduction in proinflammatory cytokines (TNF-α, IL-6 mRNA) was observed on administration of G4-PAMAM-S-S-NAC dendrimer complexes. The kits treated with NAC and G4-PAMAM-S-S-NAC showed a decrease in fetal inflammation response with improvement of motor deficits when compared to the kits that were treated with saline. The kits that were treated with G4-PAMAM-S-S-NAC conjugates bad less behavioral changes and lower microglial activation in the brain when compared to the kits that received NAC alone due to the sustained delivery of NAC from G4-PAMAM-S-S-NAC conjugate. The results indicated that G4-PAMAM-S-S-NAC conjugates have a greater effect than NAC alone since it is preferentially taken up by activated macrophages and microglial cells, reducing the inflammatory and oxidative and nitrosative effects.

Treatment with G4-PAMAM-S-S-NAC dendrimer complexes reduced white matter injury and microglia activation. A significant reduction in dose of NAC was observed when administered as G4-PAMAM-S-S-NAC to elicit the similar response as that observed for free NAC. Both free NAC at concentration 100 mg/kg and G4-PAMAM-S-S-NAC at concentration 10 mg/kg, 10 mg elicit identical responses, demonstrating that on conjugating to dendrimer a reduction in dose is achieved. G4-PAMAM-S-S-NAC at lower concentrations than free NAC shows significant protective effects against LPS-induced brain injuries, suppression of TNF-cx and down-regulation of IL-6 activity. This activity of the dendrimer-NAC conjugates may be attributed to its ability to interfere with the early inflammatory responses by blocking or modifying the signal transduction factor NF-kappa.B and nitrotyrosine, thereby modulating cellular activation.

The down-regulation of TNF-α and IL-6 in the hippocampus, is likely to be attributed to the preferential biodistribution of dendrimer 2.5 complexes with specific cell uptake by microglia cell in the brain. The dendrimer-NAC complexes can be used for treatment of pregnant women developing clinical symptoms associated with maternal infection, with increased risk of developing PVL and CP in infants. The results show that inhibition of microglial cells, astrocytes with Dendrimer-NAC decreased the white matter injury in the newborn rabbit brain. Further, the dendrimers exhibit sustained release of conjugated drugs, and enhance the effectiveness of drugs over a prolonged period. At lower dose, Dendrimer-NAC conjugates were more effective than NAC alone. The dendrimer-NAC conjugates seem to offer more advantages including significant dose reduction, enhanced bioavailability, and reduction in dosing.

6 and 8 arm PEG-NAC conjugates released 74% of NAC in the intracellular GSH concentration (2 and 10 mM), within 2 hours. At a concentration range of between 0.008-0.8 mM, the conjugates were nontoxic to the microglial cells. At an equimolar concentration of NAC(0.5 mM) the 6-arm-PEG-S-S-NAC and 8-arm-PEG-S-S-NAC were more efficient in inhibition of GSH depletion than the free NAC. Both 6 and 8-arm-PEG-S-S-NAC conjugates, each at 0.5 mM and 5 Mm concentration showed significant inhibition in ROS production when compared to free NAC at equimolar concentrations. The studies demonstrate that the conjugates are superior in inhibition of the NO production as compared to the free NAC. At the highest concentration (5 mM), the free drug reduced the H₂O₂ levels and nitrite levels by 30-40%, whereas the conjugates reduced the H₂O₂ and nitrite levels by more than 70%. This shows that the conjugates are able to traffic the drug inside the cells, and release the drug in the free form and are significantly more efficacious than the free drug. At S mM concentration 6-arm-PEG-S-S-NAC conjugate (1) showed significant inhibition (70%) of TNF-α production when compared to equivalent concentration of NAC (Pb0.05). 8-arm-PEG-S-S-NAC conjugate (3) showed significant inhibition of TNF-α production (70%) at 5 mM when compared to equivalent concentration of NAC(Pb0.05 and Pb0.01). PEGylated NAC is a dendrimer complex with utility for the pharmaceutical industry, as PEGs are approved for human use and this device addresses limitations of NAC and provides greater efficacy.

As demonstrated in the examples, six generation dendrimers provide even greater delivery, especially to damaged brain tissue. The doses determined with four generation dendrimers are adjusted accordingly to compensate for the increased delivery. One skilled in the art is able to determine the relative dosing without undue experimentation.

Typically, an attending physician will decide the dosage of the composition with which to treat each individual subject, taking into consideration a variety of factors, such as age, body weight, general health, diet, sex, compound to be administered, route of administration, and the severity of the condition being treated. The dose of the compositions can be about 0.0001 to about 1000 mg/kg body weight of the subject being treated, from about 0.01 to about 100 mg/kg body weight, from about 0.1 mg/kg to about 10 mg/kg, and from about 0.5 mg to about 5 mg/kg body weight

In general the timing and frequency of administration will be adjusted to balance the efficacy of a given treatment or diagnostic schedule with the side-effects of the given delivery system. Exemplary dosing frequencies include continuous infusion, single and multiple administrations such as hourly, daily, weekly, monthly or yearly dosing.

It will be understood by those of ordinary skill that a dosing regimen used in the inventive methods can be any length of time sufficient to treat Rett syndrome and/or related autism spectrum disorders in the subject. The term “chronic” as used herein, means that the length of time of the dosage regimen can be hours, days, weeks, months, or possibly years.

The dendrimer complexes can be administered in combination with one or more additional therapeutically active agents, which are known to be capable of treating conditions or diseases discussed above.

### B. Disorders or Diseases to be Treated

Inflammation in the brain plays a key role in the pathogenesis and worsening of symptoms in children with RTT and autism spectrum disorders. As used herein, the term “inflammatory disease of the brain” means diseases of the brain associated with activation of the microglia or astrocytes of the brain, including, for example RTT and autism spectrum disorders as classified in the Diagnostic and Statistical Manual V of the American Psychiatric Association.

### Rett Syndrome

Rett syndrome (RTT) is one example of a debilitating neurodevelopmental disorder, with many aspects common to autism spectrum disorders. RTT affects girls by slowing development followed by sudden regression in function, in children who initially appear normal. Inflammation in the brain plays a key role in the pathogenesis and worsening of symptoms in children with RTT and autism. There is no cure available for these disorders.

Children with Rett syndrome often exhibit autistic-like behaviors in the early stages. The earliest symptoms of Rett syndrome, emerging around 6 to 18 months of age, look much like autism: The children withdraw from social engagement, lose communication abilities and develop repetitive movements such as hand-wringing. Increased glutamate is seen in CSF of patients with Rett Syndrome and increased microglial activation is seen in autopsy specimens of patients with autism.

The animal model of Rett has the most common genetic abnormality associated with Rett which is MeCP2 deletion. The mice demonstrate the characteristic paw wringing and clasping movements as seen in patients with Rett and autism. In this model the animal rapidly progresses from onset of symptoms at 3 weeks to death by about 7 weeks of age.

Treatment with a Dendrimer-anti-inflammatory agent (D-NAC 10 mg/kg) once a week starting from either 1 week or 3 weeks of age results in improvement in symptoms, delayed symptom onset and/or non-progression of symptoms compared to animals that are not treated, but this is not associated with a significant increase in survival. The dendrimer-NAC treatment resulted in an increase in weight gain in the treated animals. There is also an improvement in microglial morphology and phenotype in the treated animals.

In humans, improving symptoms would be a significant advance. In a preferred embodiment, the dendrimer complex would be used to deliver an anti-inflammatory agent (D-NAC) and anti-excitotoxic and D-anti-glutamate agents. Preferred candidates are: MK801, Memantine, Ketamine, 1-MT, JHU-29, anti-glutaminase inhibitors and OCPII inhibitors such as 2-MPPA and 2-PMPA.

### Autism Spectrum Disorders

Autism spectrum disorder (ASD) is characterized by:
Persistent deficits in social communication and social interaction across multiple contexts;
Restricted, repetitive patterns of behavior, interests, or activities; Symptoms must be present in the early developmental period (typically recognized in the first two years of life); and,
Symptoms cause clinically significant impairment in social, occupational, or other important areas of current functioning.

The term “spectrum” refers to the wide range of symptoms, skills, and levels of impairment or disability that children with ASD can have. Some children are mildly impaired by their symptoms, while others are severely disabled. The latest edition of the Diagnostic and Statistical Manual of Mental Disorders (DSM-S) no longer includes Asperger's syndrome; although the characteristics of Asperger's syndrome are included within the broader category of ASD.

In some cases, babies with ASD may seem different very early in their development. Even before their first birthday, some babies become overly focused on certain objects, rarely make eye contact, and fail to engage in typical back-and-forth play and babbling with their parents. Other children may develop normally until the second or even third year of life, but then start to lose interest in others and become silent, withdrawn, or indifferent to social signals. Loss or reversal of normal development is called regression and occurs in some children with ASD.

Autism spectrum disorder (ASD) diagnosis is often a two-stage process. The first stage involves general developmental screening during well-child checkups with a pediatrician or an early childhood health care provider. Children who show some developmental problems are referred for additional evaluation. The second stage involves a thorough evaluation by a team of doctors and other health professionals with a wide range of specialties. At this stage, a child may be diagnosed as having ASD or another developmental disorder.

At this time, the only medications approved by the FDA to treat aspects of ASD are the antipsychotics risperidone (Risperdal) and aripiprazole (Abilify). These medications can help reduce irritability—meaning aggression, self-harming acts, or temper tantrums—in children ages

Some medications that may be prescribed off-label for children with ASD include the following:

Antipsychotic medications are more commonly used to treat serious mental illnesses such as schizophrenia. These medicines may help reduce aggression and other serious behavioral problems in children, including children with ASD. They may also help reduce repetitive behaviors, hyperactivity, and attention problems.

Antidepressant medications, such as fluoxetine or sertraline, are usually prescribed to treat depression and anxiety but are sometimes prescribed to reduce repetitive behaviors. Some antidepressants may also help control aggression and anxiety in children with ASD.

Stimulant medications, such as methylphenidate (Ritalin), are safe and effective in treating people with attention deficit hyperactivity disorder (ADHD). Methylphenidate has been shown to effectively treat hyperactivity in children with ASD as well. But not as many children with ASD respond to treatment, and those who do have shown more side effects than children with ADHD and not ASD.

The dendrimer conjugates described herein should have efficacy for treatment and diagnosis of such individuals, particularly in view of recent studies showing that patients with autism have evidence of neuroinflammation as seen by increased presence of activated microglia and astrocytes in post-mortem brain specimens and in CSF levels of cytokines. Vargas, et al., Ann Neurol. 2005 January; 57 (1): 67-81. Erratum in: Ann Neurol. 2005 February; 57 (2): 304.

### Excitotoxicity Disorders

Excitotoxicity is a process through which nerve cells become damaged because they are overstimulated. A number of conditions are linked with excitotoxicity including stroke, traumatic brain injury, multiple sclerosis, amyotrophic lateral sclerosis, Alzheimer's disease, and spinal injuries. Damage to the nerve cells results in corresponding neurological symptoms which can vary depending on which cells are damaged and how extensive the damage is. Once damaged, nerve cells cannot be repaired and the patient can experience permanent impairments.

The process through which excitotoxicity occurs starts with an elevation of glutamate. Glutamate is an excitatory neurotransmitter which acts to facilitate electrical signaling between nerve cells. When glutamate levels rise too much, however, they essentially jam a neuron in the open position, allowing calcium to flow freely into the cell. The calcium damages the structure and DNA of the cell, and creates a cascading reaction as cells die and release glutamate which floods neighboring cells, causing the damage to spread.

Several receptors on nerve cells are sensitized to glutamate, including the AMPA and NDMA receptors. This interaction between neurons may be either excitatory or inhibitory. The major excitatory amino acid neurotransmitters are glutamate and aspartate, while GABA (γ-aminobutyric acid), glycine (aminoacetic acid), and taurine are inhibitory.

A challenging diversity of neurologic disorders, including stroke, trauma, epilepsy, and even neurodegenerative conditions, such as Huntington disease, AIDS dementia complex, and amyotrophic lateral sclerosis, but this spectrum of disease is not usually thought of as sharing the same mechanism of neuronal injury and death. Trauma is a blunt mechanism that massively elevates the extracellular glutamate levels. Normal extracellular glutamate concentration is about 0.6 μmol/L. Substantial neuronal excitotoxic injury occurs with glutamate concentrations of 2 to 5 μmol/L.

Traumatic injury to neurons can produce disastrous results with the exposure of the normal intracellular glutamate concentrations of about 10 μmol/L to the extracellular space. Mechanical injury to a single neuron, therefore, puts all of the neighboring neurons at risk. Significant collateral injury occurs to surrounding neurons from this type of glutamate release. One recent therapeutic strategy is to immediately treat persons with injuries to the head or spinal column with glutamate receptor blockers to minimize the spread of neuronal death beyond the immediate physically disrupted neurons.

Several mechanisms of excess glutamate accumulation probably come into play in ischernia. Abnormal release of glutamate from its storage sites in neuronal vesicles is at least one factor. A feedback loop is generated as this released glutamate stimulates additional glutamate release. Ischemia also causes energy failure that impairs the reuptake by glutamate transporters. These transporters behave as symporters, which rely on the sodium gradient across cell membranes to move glutamate against ita concentration gradients into the cell. The sodium gradient, however, is maintained by an energy-dependent pump that fails in ischemia. Such failure not only affects glutamate transport out of the synaptic space but also causes the transporters to run backward, becoming a source of extracellular glutamate rather than a sink for it, Ischemia deprives the neurons of oxygen and glucose, resulting in energy failure; however, energy failure itself is not particularly toxic to neurons. Neural toxicity occurs with the resultant activation of the cascade of glutamate receptor-dependent mechanisms. If these receptors are blocked by appropriate antagonists, the neurons can survive a period of deprivation of oxygen and metabolic substrate. This is the rationale for the recent development and trial of glutamate receptor blockers to treat acute ischemic events. While an infarcted zone cannot be salvaged, the hope is to prevent surrounding damage to the at-risk adjacent penumbra.

These receptor blockers may also be critical in the developing arena of interventional and pharmacologically related attempts to reestablish perfusion to acutely ischemic areas of the brain. Tissue reperfusion and increased oxygen concentrations to ischemic areas without concurrent halting of the excitotoxic cascade either at the receptor or intracellular levels may increase rather than decrease neuronal damage by providing additional free radicals in the form of superoxide anions as well as by increasing the intracellular cytosol calcium levels by stimulating the release of mitochondrial calcium stores.

A number of drugs have been developed and used in an attempt to interrupt, influence, or temporarily halt the glutamate excitotoxic cascade toward neuronal injury. One strategy is the “upstream” attempt to decrease glutamate release. This category of drugs includes riluzole, lamotrigine, and lifarizine, which are sodium channel blockers. The commonly used nimodipine is a voltage-dependent channel (L-type) blocker. Attempts have also been made to affect the various sites of the coupled glutamate receptor itself. Some of these drugs include felbamate, ifenprodil, magnesium, memantine, and nitroglycerin. These “downstream” drugs attempt to influence such intracellular events as free radical formation, nitric oxide formation, proteolysis, endonuclease activity, and ICE-like protease formation (an important component in the process leading to programmed cell death, or apoptosis).

The present invention will be further understood by reference to the following non-limiting examples.

### Example 1: Systemic Administration of Dendrimer-Drug Conjugates to Mice with RTT

### Materials and Methods

Detailed materials and methods used in the experiments below, including protocols for making the dendrimers-Cy5 and dendrimers-drug conjugates, have been described by Kannan S et al Sci. Transl. Med., 4:130ra46 (2012) and in U.S. Pat. No. 8,889,101.

RTT mice were the Adrian Bird model available from Jackson Laboratory.

Dendrimer injection and Animal sacrifice. RTT mice were injected with dendrimer intravenously. For intravenous injections, 600 ag of D-CyS dissolved in 100 μL of sterile PBS was injected via a 30 g needle into the femoral vein after making a small incision in the femoral region. Animals infected with free Cy5 and PBS served as positive or negative controls for this study. At appropriate time points (24 hrs, 72 hrs and 21 days, and up to six weeks later) post dendrimer injections, the animals were anesthetized using ketamine/Xylazine and euthanized using a lethal dose of sodium pentobarbital. The brains were immediately removed and processed for immunohistochemistry analysis.

High Performance Liquid Chromatography (HPLC) analysis. The purity of the dendrimer-CyS conjugates (D-Cy5) were analyzed using a Waters HPLC instrument (Waters Corporation, Milford, Massachusetts) equipped with Waters In-line degasser, binary pump, photodiode array (PDA) detector, multi fluorescence A detector and auto sampler (maintained at 4° C.) interfaced with Empower software. The HPLC chromatogram was monitored simultaneously for absorbance at 210 nm for dendrimer and 650 nm for Cy5 using Waters 2998 PDA detector and fluorescence with excitation at 645 nm and emission at 662 nm using Waters 2475 fluorescence detector. The water/acetonitrile (0.1% w/w TEA) was freshly prepared, filtered, degassed, and used as a mobile phase. TSK-Gel ODS-80 Ts (250×4.6 mm, 25 cm length with 5 μm particle size) connected to TSK-Gel guard column was used. A gradient flow was used with initial condition being 90:10 (H2O/ACN) and then gradually increasing the acetonitrile concentration to 10:90 (H2O/ACN) in 30 min and returning to original initial condition 90:10 (H2O/ACN) in 60 min with flow rate of 1 ml/min.

### Assessment of Animals and Inflammation

Weight and behavior were also assessed. Cytokines were measured using standard mice primers for the assessment of inflammatory markers (Kannan S et al Sci. Transl. Med., 4:130ra46 (2012)).

Immunohistochemistry and confocal microscopy. Brain slices were fixed in 2% paraformaldehyde (PFA) in PBS. The brains were frozen in 20% sucrose with optimum cutting temperature compound (OCT)(Sakura Finetek USA Inc., Torrance, CA) in a 1:2 ratio respectfully using dry ice in isopentane. Cryoblocks are stored at −80° C. until sectioned. Eight μm sections were cut from frozen blocks using a cryostat. Sections were incubated in rabbit anti-Ionised Calcium Binding Adapter 1 molecule (Iba-1) (Wako chemicals, USA), which is a microglia cell marker, and a goat anti-rabbit-Cy3 secondary antibody applied. Sections were analyzed on a Zeiss 510 confocal microscope. Excitation and emission wavelengths and laser settings were identical to analyze all tissue in IV injected animals. Z-stacks of sections were taken and collapsed to give an image through the depth of the whole section.

Conjugation of dendrimer conjugates. The conjugation of dendrimers to Cy5 was done using previously reported methods (Kannan et al., Science Trans. Med (April 2012). For drug experiments, dendrimers were conjugated to N-acetyl-cysteine and administered at doses ranging from 2-20 mg/kg at differing time points.

The mice were injected with D-drug or PBS every 3-4 days, Statistical analysis. The data was analyzed for the reproducibility using Student's t-test to determine the significance between two groups. A p-value equal to or less than 0.05 was considered significant.

### Results

Dendrimer conjugates can accumulate in the brain in activated 1.5 microglia which mediate inflammation, Cy5-labeled dendrimer was administered systemically at 3 weeks of age in symptomatic RTT mice, and brains were harvested, perfused, and fixed to look at dendrimer localization in microglia.

Dendrimer localized in microglia in regions of the brain where prior studies have shown injury or damage. Healthy control mice show no accumulation in the brain.

The dendrimer-drug conjugates (D)-drug), when administered systemically in mice presenting with symptoms representative of RTT, show significant improvement in overall pup health, appearance, and behavioral hallmarks of the disease by 8 weeks old, compared to non-treated with similar disease severity: Dendrimers conjugated to Cy5 administered systemically at 3 weeks of age accumulates in microglia in the lateral cortex of RTT mice.

The dendrimer-drug (D-drug) conjugate, when administered systemically every 3-4 days, starting at 3 weeks old in symptomatic RTT mice, provided significant improvement in overall health and appearance at 8 weeks old. PBS treated mice showed severe paw clenching, hunched posture, and blindness.

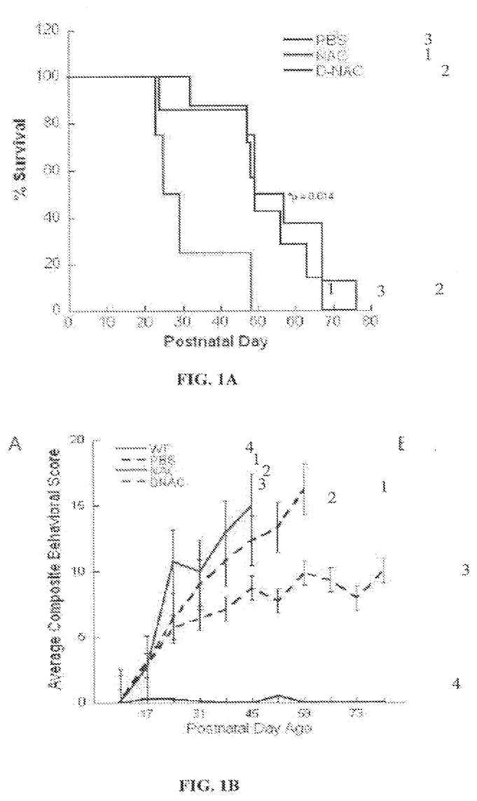

The treated mice showed improvement in survival compared to free drug (FIG. 1A). FIG. 1A is a Kaplan-Meier survival curve following NAC and D-NAC therapy in MeCP2-mill mice. Survival was assessed following twice weekly NAC or D-NAC therapy in MeCP2-pull pups. D-NAC does not improve survival compared to non-treated animals. D-NAC does improve safety of NAC. D-NAC and PBS treated MeCP2-null pups bad a significantly better 50% survival compared to NAC treated pups (p=0.014), indicating the potential toxicity of NAC when given as a free formulation. Free unconjugated drug (NAC) actually led to worse survival than non-treated Rett mice, at a comparable dose to the drug on the dendrimer-drug conjugate (DNAC). Treatment with dendrimer-drug conjugate maintained significantly improved behavior compared to PBS treated Rett mice (FIG. 1B). FIG. 1B is a graph of neurobehavioral outcomes following D-NAC therapy in MeCP2-null mice. MeCP2-null mice were treated with saline (PBS, black dashed line), 10 mg/kg NAC(red line), or 10 mg/kg (on a NAC basis) D-NAC(blue line) starting at 3 weeks of age (PND21). Pups were treated twice weekly. Behavior tests were performed at PND10 and PND17 to determine a baseline, and performed prior to treatment on each treatment day starting at PND21. Litter matched WT pups (solid black line) were used as both weight and behavioral controls. D-NAC therapy significantly improved behavioral outcome compared to NAC and PBS treatments. D-NAC improved overall appearance of MeCP2-pull mice compared to non-treated pups. Non-treated pups were significantly emaciated, bad multiple clenched paws, hunched posture, and poor eye condition.

Animals were videotaped prior to treatment every 3-4 days, and mobility, gait, tremors, paw clenching, paw clenching time, paw wringing, and respiration were all scored on a scale of 0-3, where ‘0’ indicates the worst score and ‘3’ is best or normal. A composite score was generated (range of 0-21, with normal, healthy mice having a score of 20-21) and compared among the groups, with lower scores indicate worsening behavior. Scores were averaged across all mice in the study that demonstrated similar survival (66 days old, or 6 weeks of treatment).

Brain uptake and cellular localization in WT and MeCP2-null mice was determined and compared. In the pre-symptomatic period (1 week of age), dendrimer (D-Cy5, red) localization is primarily in the supraventricular region in microglia (Iba+) and not in astrocytes (GFAP). By 7 weeks of age, well into the symptomatic period, D-Cy5 is localized in microglia in the cortex and in astrocytes in the supraventricular region. D-Cy5 remained localized in blood vessels in WT mice at both ages.

Microglia morphology was assessed in WT and MeCP2-null mice. In MeCP2-null mice (KO), microglia (Iba+) are amoevoid at 1 week of age in the regions around the ventricle. Microglia in KO mice at 2 weeks and 5 weeks of age have fewer and thinner processes, and at 7 weeks of age have more processes, but are less connected compared to WT microglia at 7 weeks.

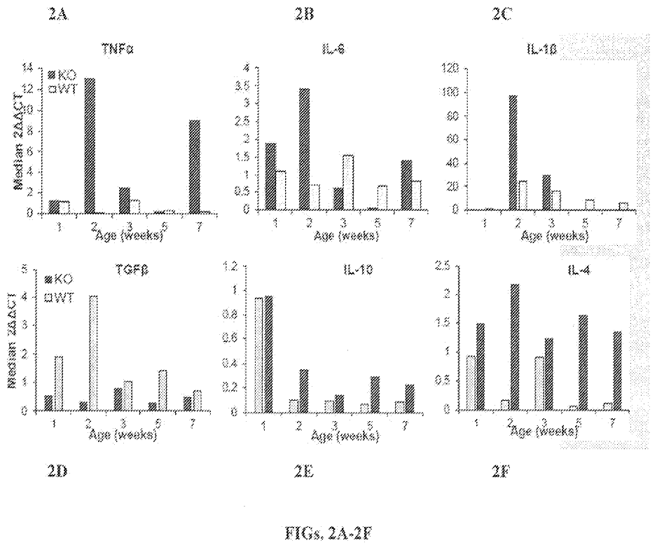

The inflammatory profile in the brains of WT and pre-symptomatic and symptomatic MeCP2-null mice was measured (FIGS. 2A-2F). mRNA levels of pro and anti-inflammatory cytokines were measured at ages 1, 2, 3, 5, and 7 weeks old in the brains of WT (grey) and MeCP2-null (black) pups.

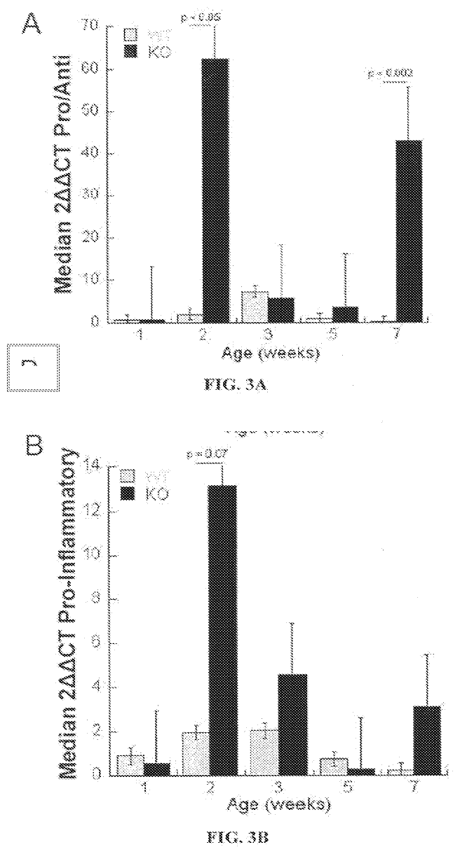

Median 2ΔΔCT values are presented, and error bars are represented by the upper and lower interquartile range. (FIG. 3A) Changes in the inflammatory profile over time are presented as a ratio of a composite pro-inflammatory score, including TNFα, IL-6, and IL-18, to a composite anti-inflammatory score, including TGF-β, IL-10, and IL-4. The composite score was generated by taking the median of all pro-inflammatory 2ΔΔCT values or all anti-inflammatory 2ΔΔCT values at each age for all pups at that age in a given genotype. (FIG. 3B) The pro-inflammatory profile in MeCP2-null mice trends towards an increase in pro inflammatory markers at 2 weeks and 7 weeks. However, the anti-inflammatory mRNA expression (FIG. 3C) shows a significant decrease in MeCP2-mill mice compared to age- and litter-matched WT mice at 2 weeks, 5 weeks, and 7 weeks of age. This suggests that the neuroinflammatory processes in the MeCP2-null mouse are driven by a decrease in anti-inflammatory expression, rather than an increase in pro-inflammatory expression.

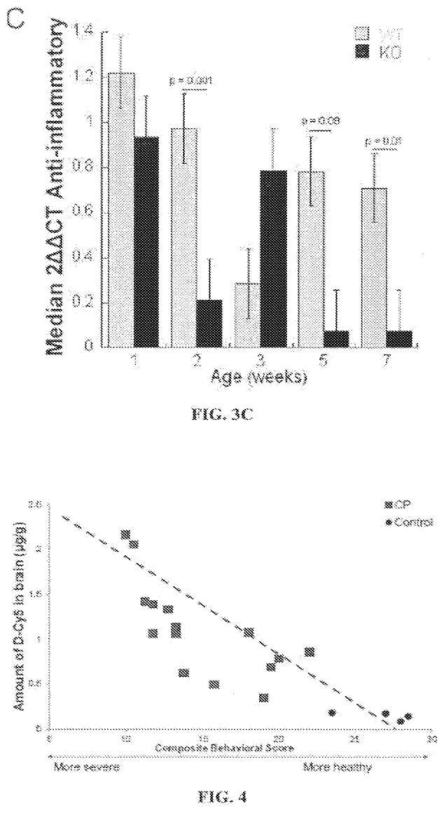

FIG. 4 is a graph of amount of D-Cy5 in brain (μg/g) as a function of severity of brain injury, based on composite behavioral score. This demonstration of correlation of uptake with severity of injury provides a means to diagnose the extent of injury.

### Example 2: Treatment of Brain Injury in Canine Model

### Materials and Methods

Dendrimer brain uptake and targeted therapy for brain injury in a canine animal model of hypothermic circulatory arrest is described by Manoj, et al., ACS Nano, 2014, 8 (3), pp 2134-2147.

Generation-6, primary hydroxyl-functionalized PAMAM dendrimers with ethylenediamine (EDA) core were used in these studies.

### Preparation of the Conjugates

Conjugates were prepared as described above.

### Canine HCA Model and Experimental Design

All experiments used a canine model of HCA developed in by the Baumgartner laboratory. (Redmond, et al., Ann. Thorac. Surg. 1995, 59, 579-584; Redmond, et al. Thorac, Cardiovase. Surg. 1994,107, 776-786) This large animal model takes advantage of certain inherent physiologic similarities between humans and canines to develop a readily translatable therapeutic model to address the neurologic injury associated with hypothermic circulatory arrest. Because this is a large animal model, one is able to replicate surgical procedures with impressive fidelity to that experienced in human operating rooms and are able to replicate a degree of neurologic injury similar to that seen in the worst human cases.

Conditioned, heartworm-negative, 6-12 month old, male, class-A dogs (approximately 30 kg) were used for all experiments (Marshal Bioresources, North Rose, NY). Experiments were approved by The Johns Hopkins University School of Medicine Animal Care and Use Committee and complied with the “Guide for the Care and Use of Laboratory Animals” (1996, U.S. National Institutes of Health).

Dogs were administered methobexital sodium (12 mg/kg IV, in divided doses), endotracheally intubated, and maintained on isoflurane inhalational anesthesia (0.5-2.0%), 100% oxygen, and IV fentanyl(150-200 μg/dose), and midazolam (2.5 mg/dose). Tympanic membrane, esophageal, and rectal probes monitored temperatures throughout the experiment. A left femoral artery cannula was placed prior to the initiation of CPB for monitoring blood pressure and sampling of arterial blood gases. EKG was continuously monitored. The right femoral artery was cannulated and the cannula advanced into the descending thoracic aorta. Venous cannulae were advanced to the right atrium from the right femoral and right external jugular veins. Closed chest CPB was initiated, and the animals were cooled. Pump flows of 60-100 ml/kg/min maintained a mean arterial pressure of 60-80 mmHg. Once tympanic temperatures reached 18° C., the pump was stopped and blood was drained by gravity into the reservoir. Dogs underwent 2 h HCA with standard hemodilution and alpha-stat regulation of arterial blood gases. After HCA, CPB was restarted and the animals were rewarmed to a core temperature of 37° C. over the course of 2 h. If sinus rhythm did not return spontaneously, the heart was defibrillated at 32° C. Serial blood gas levels were taken to ensure adequate pH and verify electrolyte concentrations, and continuous hemodynamic measurements were recorded utilizing an arterial cannula. At 37° C., each dog was weaned from CPB and the cannulae were removed. Dogs recovered from anesthesia while intubated, with frequent monitoring of vital signs, arterial blood gases, and urine output. Some animals required hemodynamic support and correction of acidosis at this stage to enable successful weaning from bypass. Once hemodynamically and clinically stable, dogs were extubated and transferred to their cages for recovery and survival, with neurologic assessments at 24 h intervals until the desired end point (24 or 72 h after bypass).

### Dendrimer Administration (for Biodistribution Studies)

Dendrimer-fluorophore conjugates were injected as a one-time bolus 24 h after hypothermic circulatory arrest. Three dogs were concurrently treated with intravenous infusion of D-FITC (140 mg per animal, approximately 5 mg/kg) and intracisterna magna (ICM, “into the brain”) injection of D-Cy5 (5 mg per animal, 0.17 mg/kg) and euthanized 48 h post-conjugate administration. Tissue uptake and biodistribution were subsequently measured at sacrifice (48 h after administration). Since FITC and Cy were analyzed at their distinct characteristic wavelengths, their biodistribution could be assessed simultaneously.

### Dendrimer Administration (for Efficacy Studies)

Free drugs (VPA and NAC) or dendrimer-drug conjugates were administered intravenously before and after HCA. Doses for free drug administration were based on our previous studies in which neuroprotection was achieved with free VPA and based on the literature for free M-acetylcysteine. Previous studies have reported that pretreatment with NAC is protective in models of cardiac arrest. Doses for the dendrimer-drug conjugates were set at 1/10 (VPA) or 1/30 (NAC) of the free drug doses, based on prior findings of striking neuroprotection at such dose ratios in the rabbit CP model. For the free drugs, animals were treated with 100 mg/kg of VPA and 300 mg/kg of NAC, of which half the dose was administered intravenously prearrest and the rest was administered postarrest. For the dendrimer-drug conjugates, dogs were treated intravenously with D-NAC containing 10 mg/kg of NAC and/or D-VPA with 10 mg/kg of VPA. D-VPA was administered intravenously as a 25% bolus prior to HCA, followed by 75% infusion over 2 h after HCA was completed, D-NAC was intravenously administered as a 50% bolus pre-HCA and a 50% infusion over 2 b after HCA was complete. These regimens are similar to what was used for free drugs.

### Euthanasia

Animals were euthanized by exsanguination. After sedation and intubation, animals underwent median sternotomy and cannulation of the ascending aorta using a 22-French cannula. CPB was initiated after clamping the descending aorta to ensure the brain was perfused with 12 L of ice-cold saline (4° C.) at 60 mmHg. The right atrial appendage was transected, and the venous return was allowed to drain. Brains were harvested immediately after perfusion, hemispheres were separated, and one hemisphere was fixed in 10% neutral buffered formalin (for immunohistochemical evaluation and imaging) while the other hemisphere was cut into 1 cm coronal slices and rapidly frozen (for biodistribution quantification).

### Fluorescence Microscopy

Cryostat sections of hippocampus and cerebellum were mounted with antifade media (ProLong Gold with DAPI, Molecular Probes, Inc., Eugene, OR). Fluorescence images were obtained using a Zeiss Axiolmager M2, with equal exposure times for all samples of each brain region. To optimize image contrast and brightness, display settings were adjusted equally within each set of images.

### Neurologic Evaluation

Clinical neurologic assessment was performed on all animals every 24 h until sacrifice. The dog-specific behavior scale used in this study was validated at the International Resuscitation and Research Center, University of Pittsburgh School of Medicine. There were five components of neurologic function evaluated: level of consciousness, respiratory pattern, cranial nerve function, motor and sensory function, and behavior. Two investigators independently assigned each component a score between 0 (normal) and 100 (severe injury), and these were averaged and summed to obtain the total 2.5 score, with a possible range from 0 (normal) to 500 (brain death).

### Results

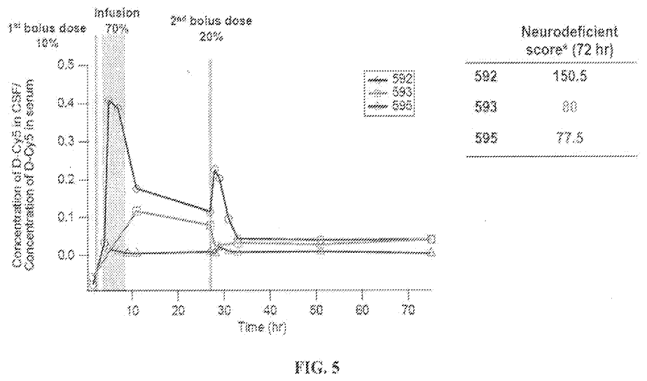

G6 PAMAM dendrimers are superior to G4 dendrimers to deliver drugs across the injured BBB as demonstrated in a canine model of hypothermic circulate cardiac arrest induced brain injury. G6 dendrimers maintained high cerebral spinal fluid (CSF) to serum ratio over a sustained period of time. Maintaining such a high CSF/serum ratio is a key stumbling block for many CNS drugs. See FIG. 5. The high CSF levels seen in the Injured brain is a key new feature. Accumulation of dendrimers is dependent of the extent of injury (see FIG. 4), based on studies showing G6 dendrimers are internalized by activated microglia and injured neurons (ACS Nano. 2014 Mar. 25; 8 (3): 2134-47.)

As shown in FIG. 5, G6 dendrimers have a high partition in Cerebrospinal Fluid (CSF), with CSF/Serum ratio higher than 10% for Dog 592 and 593 until 24 hours and ˜4.5% at 72 hours. During and shortly after the infusion time, the ratio can go as high as 40% depending on the extent of injury.

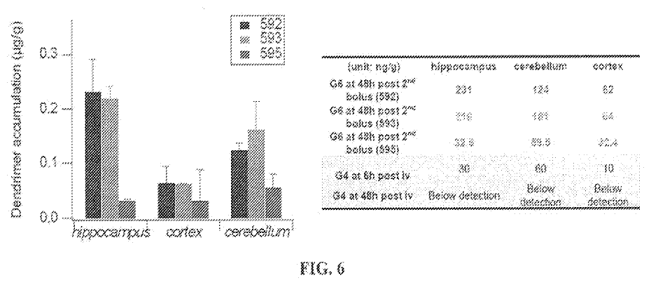

As shown in FIG. 6, the brain accumulation of G6 dendrimers is region dependent, with highest accumulation in hippocampus, following with cerebellum and cortex, consistent with the pattern of injury.

At 48 hours post dendrimer administration, G6 dendrimers showed significant higher brain accumulation than G4 dendrimer (below detection limit) across all regions in the brain. See FIG. 6. The levels of G6 dendrimer in the injured regions, oven at 48 hours after administration, is many fold higher than that of the (4 dendrimers at early time of 6 hours.

In the hippocampus, G6 dendrimers showed higher accumulation in dentate gyrus than CA1 and CA3 region. In the hippocampus, 06 dendrimers show different types of cellular localization, with uptake mainly by activated microglia and injured neurons

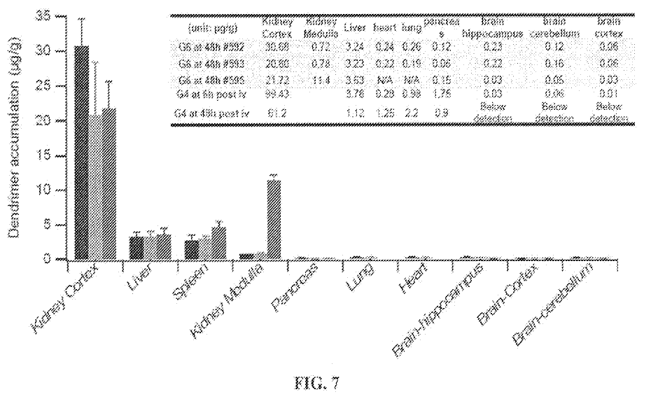

As shown by FIG. 7, G6 dendrimer mainly accumulated in kidney cortex and liver at 48 hours post 2nd bolus dose, suggesting renal and hepatic clearance are both important for the dendrimer removal from circulation. Compared to G4 dendrimers, G6 dendrimers show lower kidney levels, consistent with higher serum levels.

The results demonstrate that neither 04 nor G6 dendrimer is toxic at 500 fold higher doses, and is cleared intact via the kidney.

Modifications and variations of the methods and materials described herein will be apparent to those skilled in the art and are intended to be encompassed by the claims.

1-23. (canceled)

24. A method of detecting neuroinflammation in a subject, the method comprising:
administering to the subject a composition comprising a hydroxyl-terminated poly(amidoamine)(PAMAM) dendrimer conjugated to an imaging agent,
wherein the imaging agent comprises fluorine-18 (F-18); and
detecting the imaging agent in a tissue of the central nervous system in the subject,
wherein the detected imaging agent is indicative of neuroinflammation in the subject.

25. The method of claim 24, wherein the PAMAM dendrimer is a generation 4, generation 5, or generation 6 dendrimer.

26. The method of claim 25, wherein the PAMAM dendrimer is a generation 4 dendrimer.

27. The method of claim 24, wherein the composition does not comprise a targeting agent.

28. The method of claim 24, wherein the PAMAM dendrimer is conjugated to the imaging agent via a linker comprising polyethylene glycol.

29. The method of claim 24, wherein the tissue of the central nervous system in the subject comprises brain tissue.

30. The method of claim 24, wherein the subject has a neurological or neurodegenerative disorder.

31. The method of claim 24, wherein the subject has a disease or disorder selected from the group consisting of multiple sclerosis, amyotrophic lateral sclerosis, Alzheimer's disease, Parkinson's disease, and Huntington's disease.

32. The method of claim 24, wherein the composition is administered to the subject systemically.

33. The method of claim 32, wherein the composition is administered to the subject intravenously.

34. A method of detecting neuroinflammation in a subject, the method comprising:
administering to the subject a composition comprising a hydroxyl-terminated poly(amidoamine)(PAMAM) dendrimer conjugated to an imaging agent,
wherein the imaging agent comprises a chelator and copper-64 (64Cu); and
detecting the imaging agent in a tissue of the central nervous system in the subject,
wherein the detected imaging agent is indicative of neuroinflammation in the subject.

35. The method of claim 34, wherein the PAMAM dendrimer is a generation 4, generation 5, or generation 6 dendrimer.

36. The method of claim 35, wherein the PAMAM dendrimer is a generation 4 dendrimer.

37. The method of claim 34, wherein the PAMAM dendrimer is conjugated to the imaging agent via a linker comprising polyethylene glycol.

38. The method of claim 34, wherein the chelator is selected from the group consisting of di-ethylene tri-amine penta-acetic acid (DTPA), 1,4,7,10-tetra-azacyclododecane-1,4,7,10-tetraacetic acid (DOTA), di-amine dithiol, activated mercaptoacetyl-glycyl-glycyl-glycine (MAG3), and hydrazidonicotinamide (HYNIC).

39. The method of claim 34, wherein the tissue of the central nervous system in the subject comprises brain tissue.

40. The method of claim 34, wherein the subject has a neurological or neurodegenerative disorder.

41. The method of claim 34, wherein the subject has a disease or disorder selected from the group consisting of multiple sclerosis, amyotrophic lateral sclerosis, Alzheimer's disease, Parkinson's disease, and Huntington's disease.

42. The method of claim 34, wherein the composition is administered to the subject systemically.

43. The method of claim 42, wherein the composition is administered to the subject intravenously.
